(19) United States
(12) Patent Application Publication
(10) Pub. No.: US 2026/0210954 A1
(43) Pub. Date: Jul. 23, 2026

(54) SPR DEVICE

(71) Applicant: AFFINITY SENSING TECHNOLOGY CO., LTD., Taipei City (TW)

(72) Inventors: Chih-Han CHEN, Taipei City (TW); Yu-Chun LIN, Taipei City (TW)

(21) Appl. No.: 19/442,477

(22) Filed: Jan. 7, 2026

(30) Foreign Application Priority Data
Jan. 20, 2025 (TW) 114102375

(51) Int. Cl. G01N 33/543; G01N 21/552

(52) U.S. Cl. CPC G01N 33/54373; G01N 21/553; G01N 2201/0221; G01N 2201/0636

(57) ABSTRACT

An SPR device including: a detection platform having a detection area and an optical component affixed to a bottom surface of the detection area; a light source for generating an incident light; and an optical detector for receiving an output light; where the optical component includes an integrated module having an incident light guiding structure, a reflective section and a reflected light guiding structure, the reflective section being attached to the bottom surface, where the incident light guiding structure includes a first mirror assembly for guiding the incident light to the reflective section along an oblique incident path to irradiate the detection area and thereby generate a reflected light from the detection area; and the reflected light guiding structure includes a second mirror assembly for altering the direction of the reflected light to provide the output light.

### BACKGROUND OF THE INVENTION

### Field of the Invention

The invention relates to molecular detection technology, particularly to an SPR (surface plasmon resonance) device for detecting molecules in microfluidics.

### Description of the Related Art

The commonly used molecular detection method is the enzyme-linked immunosorbent assay (ELISA). However, the method has the drawbacks of requiring a high-cost and bulky ELISA instrument, and being unable to get the detection result on-site in real time.

Moreover, during on-site molecular detection of contaminants present in water sources, food, or food products, where the contaminants include haptenic substance such as pesticides, antibiotics, malachite green, preservatives, insecticides, or melamine, the commonly employed rapid test strips exhibit limitations such as low sensitivity and inability to provide a quantized detection result.

To address the aforementioned issues, a novel molecular detection solution is needed in the field.

### SUMMARY OF THE INVENTION

The primary objective of the invention is to provide an SPR (surface plasmon resonance) device that, through integrating optical components into a compact module to facilitate miniaturization and portability of the SPR device, is convenient for the operator to perform molecular detection on a microfluidic sample.

Another objective of the invention is to provide an SPR device capable of generating a real-time molecular detection result of a microfluidic sample.

To attain the aforementioned objectives, an SPR device is proposed, including:

a detection platform having a detection area and an optical component affixed to a bottom surface of the detection area, the optical component having a light-entering side and a light-exiting side for receiving an incident light and providing an output light correspondingly;

a light source for generating the incident light; and

an optical detector for receiving the output light;

where the optical component includes an integrated module having an incident light guiding structure, a reflective section and a reflected light guiding structure, the reflective section being attached to the bottom surface, where the incident light guiding structure includes a first mirror assembly for guiding the incident light to the reflective section along an oblique incident path to irradiate the detection area and thereby generate a reflected light from the detection area; and the reflected light guiding structure includes a second mirror assembly for altering the direction of the reflected light to provide the output light.

In one embodiment, the output light travels substantially in the same direction as the incident light or substantially in the opposite direction from the incident light.

In one embodiment, the first mirror assembly includes a plane mirror and a curved mirror disposed obliquely opposite each other, or two plane mirrors disposed obliquely opposite each other.

In one embodiment, the second mirror assembly includes a plane mirror and a curved mirror disposed obliquely opposite each other, or two plane mirrors disposed obliquely opposite each other.

In one embodiment, the reflective section includes a light-transmissive member and an SPR excitation layer covering the light-transmissive member. The light-transmissive member is a glass layer or a plastic glass layer. The SPR excitation layer includes a metal film, where the metal film may include at least one film selected from a group consisting of a gold film, a silver film, an aluminum film, and a copper film.

In one embodiment, an oxidation-resistant film is further formed on the surface of the metal film.

In one embodiment, the SPR device further includes a processor. When in operation, a microfluidic chip containing a microfluidic sample to be tested is installed in the detection area. The processor obtains a signal parameter of the reflected light from the optical detector and performs a conversion operation on the signal parameter to determine at least one physical or chemical characteristic value of at least one type of molecules in the microfluidic sample, where the conversion operation is a lookup table operation.

For possible embodiments, the at least one type of molecules can be proteins, small molecules, nucleic acids, bioactive molecules, intact cells, viral particles, or bacteria.

For possible embodiments, the at least one physical or chemical characteristic value can include the concentration of one type of the at least one type of molecules and/or the binding rate constant (Ka) or dissociation rate constant (Kd) between two types of the at least one type of molecules.

For possible embodiments, the signal parameter can be a light intensity parameter, a light phase parameter, a resonance angle parameter, a resonance wavelength parameter, or an interference pattern parameter.

To make it easier for our examiner to understand the objective of the invention, its structure, innovative features, and performance, we use preferred embodiments together with the accompanying drawings for the detailed description of the invention.

### BRIEF DESCRIPTION OF THE DRAWINGS

FIG. 1 illustrates an embodiment of the SPR device of the invention;

FIG. 2 illustrates an embodiment of an optical component of the SPR device shown in FIG. 1;

FIG. 3 illustrates another embodiment of the optical component of the SPR device shown in FIG. 1;

FIG. 4 illustrates another embodiment of the SPR device of the invention;

FIG. 5 illustrates an embodiment of an optical component of the SPR device shown in FIG. 4;

FIG. 6 illustrates an embodiment of a reflected light guiding structure of the optical component shown in FIG. 5; and

FIG. 7 illustrates another embodiment of a reflected light guiding structure of the optical component shown in FIG. 5.

### DETAILED DESCRIPTION OF THE PREFERRED EMBODIMENTS

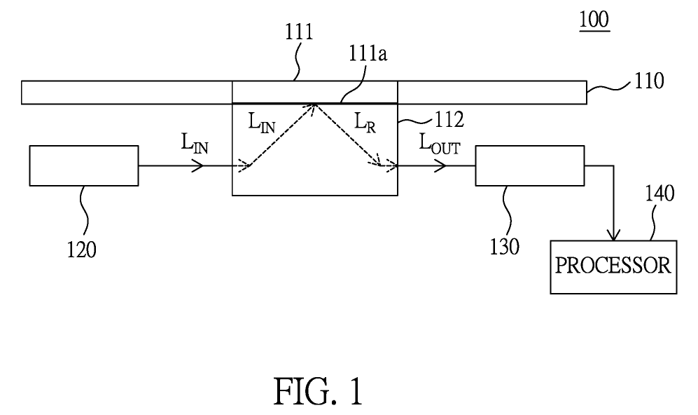

Please refer to FIG. 1, which illustrates an embodiment of the SPR device of the invention. As shown in FIG. 1, an SPR device 100 includes a detection platform 110, a light source 120, an optical detector 130, and a processor 140 for performing a molecular detection procedure.

The detection platform 110 includes a detection area 111 and an optical component 112 affixed to a bottom surface 111a of the detection area 111. The optical component 112 has a light-entering side and a light-exiting side for receiving an incident light L_{IN} and providing an output light L_{OUT} correspondingly. In this embodiment, the output light L_{OUT} travels substantially in the same direction as the incident light L_{IN}.

The light source 120 is used to generate the incident light L_{IN}; the optical detector 130 is used to receive the output light L_{OUT}.

To be specific, the optical component 112 includes an integrated module having an incident light guiding structure, a reflective section and a reflected light guiding structure, where the reflective section is affixed to the bottom surface 111a; the incident light guiding structure includes a first mirror assembly for guiding incident light L_{IN} toward the reflective section along an oblique incident path to irradiate the detection area 111 and thereby generate a reflected light L_{R} from the detection area 111; and the reflected light guiding structure includes a second mirror assembly for altering the direction of the reflected light L_{R} to provide the output light L_{OUT}. Notably, integrating the incident light guiding structure, the reflective section, and the reflected light guiding structure into a single module enables the optical component 112 to become a compact optical component, thereby facilitating miniaturization and portability of the SPR device.

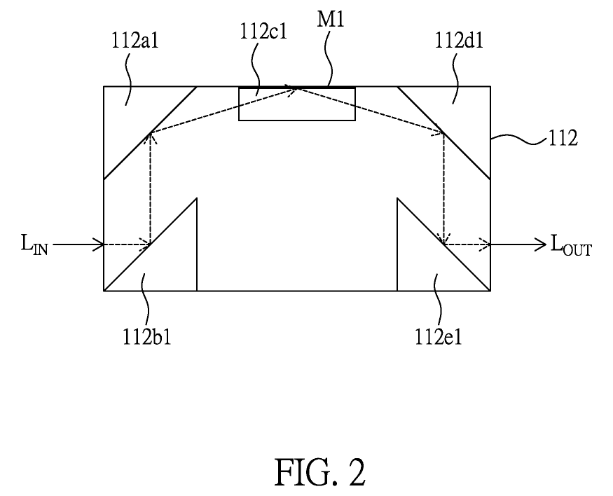

Please refer to FIG. 2, which illustrates an embodiment of the optical component 112 of the SPR device 100 shown in FIG. 1. As shown in FIG. 2, the first mirror assembly of the optical component 112 includes two plane mirrors 112a1 and 112b1 disposed obliquely opposite each other; the second mirror assembly includes two plane mirrors 112d1 and 112e1 disposed obliquely opposite each other; and the reflective section includes a light-transmissive member 112c1 and an SPR excitation layer M1 covering the light-transmissive member 112c1, where the light-transmissive member 112c1 can be a glass layer or a plastic glass layer; the SPR excitation layer M1 can include a metal film, and the metal film can include at least one layer of film selected from a group consisting of a gold film, a silver film, an aluminum film, and a copper film, that is, the metal film can be a single-layer metal film or a multi-layer metal film, and the multi-layer metal film can be composed of different metal films selected from the group. Additionally, an oxidation-resistant film can be further formed on the surface of the metal film to prevent oxidation of the metal film.

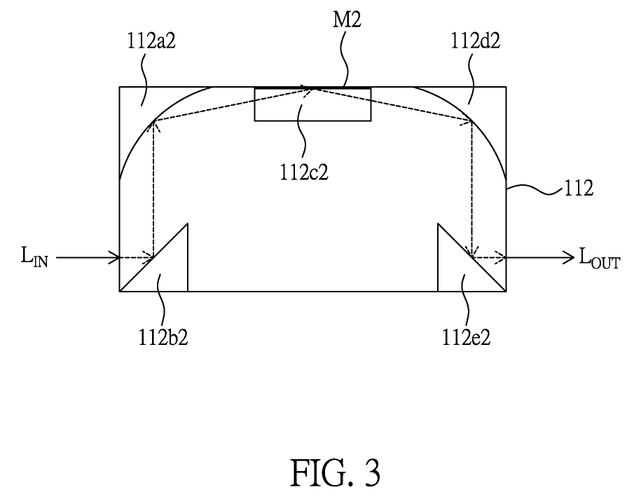

In addition, the first mirror assembly and the second mirror assembly can also be implemented with a plane mirror and a concave mirror disposed obliquely opposite each other. Please refer to FIG. 3, which illustrates another embodiment of the optical component 112 of the SPR device 100 shown in FIG. 1. As shown in FIG. 3, the first mirror assembly of the optical component 112 includes a concave mirror 112a2 and a plane mirror 112b2 disposed obliquely opposite each other, and the second mirror assembly includes a concave mirror 112d2 and a plane mirror 112e2 disposed obliquely opposite each other. In this embodiment, the concave mirrors 112a2 and 112d2 provide a fine-tuning function for the optical path angle.

To be specific, the molecular detection procedure includes:

(1) mounting a microfluidic chip in the detection area 111, where the microfluidic chip contains a microfluidic sample to be detected;

(2) driving the light source 120 to generate the incident light L_{IN};

(3) driving the optical detector 130 to receive the output light L_{OUT} to obtain a signal parameter of the reflected light L_{R}, where the signal parameter can be a light intensity parameter, a light phase parameter, a resonance angle parameter, a resonance wavelength parameter, or an interference pattern parameter; and

(4) utilizing the processor 140 to obtain the signal parameter from the optical detector 130 and perform a conversion operation on the signal parameter to determine at least one physical or chemical characteristic value of at least one type of molecules of the microfluidic sample, where the conversion operation is a lookup table operation, and a lookup table required for the lookup table operation is stored in a memory; the at least one type of molecules can be proteins, small molecules, nucleic acids, bioactive molecules, intact cells, viral particles, or bacteria; and the at least one physical or chemical characteristic value can include the concentration of one type of the at least one type of molecules and/or the binding rate constant (Ka) or dissociation rate constant (Kd) between two types of the at least one type of molecules.

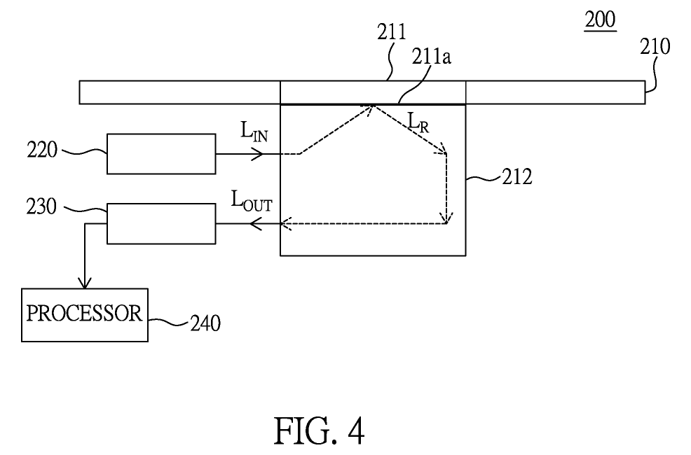

Please refer to FIG. 4, which illustrates another embodiment of the SPR device of the invention. As shown in FIG. 4, an SPR device 200 includes a detection platform 210, a light source 220, an optical detector 230, and a processor 240 for performing a molecular detection procedure.

The detection platform 210 includes a detection area 211 and an optical component 212 affixed to a bottom surface 211a of the detection area 211. The optical component 212 has a light-entering side and a light-exiting side for receiving an incident light L_{IN} and providing an output light L_{OUT} correspondingly. In this embodiment, the output light L_{OUT} travels substantially in the opposite direction from the incident light L_{IN}.

The light source 220 is used to generate the incident light L_{IN}; the optical detector 230 is disposed on the same side as the light source 220 to receive the output light L_{OUT}.

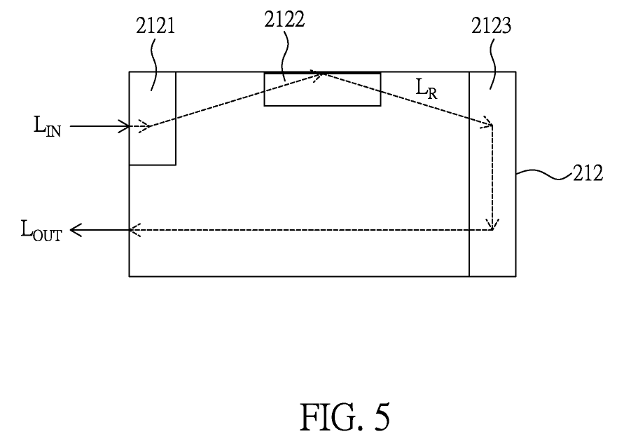

Please refer to FIG. 5, which illustrates an embodiment of the optical component 212 of the SPR device 200 shown in FIG. 4. As shown in FIG. 5, the optical component 212 includes an integrated module having an incident light guiding structure 2121, a reflective section 2122 and a reflected light guiding structure 2123, where the reflective section 2122 is affixed to the bottom surface 211a; the incident light guiding structure 2121 includes a first mirror assembly for guiding incident light L_{IN} toward the reflective section 2122 along an oblique incident path to irradiate the detection area 211 and thereby generate a reflected light L_{R} from the detection area 211; and the reflected light guiding structure 2123 includes a second mirror assembly for altering the direction of the reflected light L_{R} to provide the output light L_{OUT}. Notably, integrating the incident light guiding structure 2121, the reflective section 2122, and the reflected light guiding structure 2123 into a single module enables the optical component 212 to become a compact optical component, thereby facilitating miniaturization and portability of the SPR device.

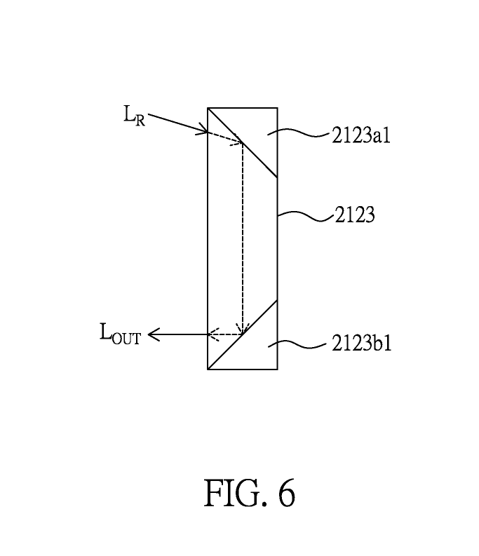

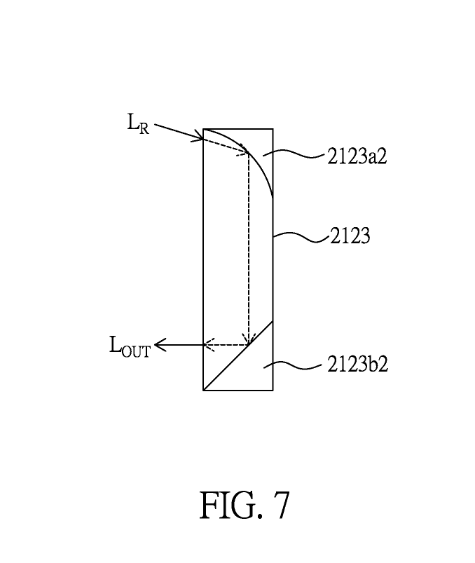

Additionally, please refer to FIGS. 6 and 7, which illustrate two embodiments of the reflected light guiding structure 2123 of the optical component 212 shown in FIG. 5. As shown in FIG. 6, the reflected light guiding structure 2123 includes two plane mirrors 2123a1 and 2123b1 disposed obliquely opposite each other. As shown in FIG. 7, the reflected light-guiding structure 2123 includes a concave mirror 2123a2 and a plane mirror 2123b2 disposed obliquely opposite each other.

With the designs disclosed above, the invention offers the advantages as follows:

A. The SPR device of the invention makes it convenient for the operator to perform molecular detection on a microfluidic sample by integrating optical components into a compact module to realize a small sized and portable SPR device; and

B. The SPR device of the invention is capable of generating a real-time molecular detection result of a microfluidic sample.

While the invention has been described by way of example and in terms of preferred embodiments, it is to be understood that the invention is not limited thereto. On the contrary, it is intended to cover various modifications and similar arrangements and procedures, and the scope of the appended claims therefore should be accorded the broadest interpretation so as to encompass all such modifications and similar arrangements and procedures.

In summation of the above description, the present invention herein enhances the performance over the conventional structure and further complies with the patent application requirements and is submitted to the Patent and Trademark Office for review and granting of the commensurate patent rights.

What is claimed is:

1. An SPR device, comprising:
a detection platform having a detection area and an optical component affixed to a bottom surface of the detection area, the optical component having a light-entering side and a light-exiting side for receiving an incident light and providing an output light correspondingly;
a light source for generating the incident light; and
an optical detector for receiving the output light;
wherein the optical component includes an integrated module having an incident light guiding structure, a reflective section and a reflected light guiding structure, the reflective section being attached to the bottom surface, wherein the incident light guiding structure includes a first mirror assembly for guiding the incident light to the reflective section along an oblique incident path to irradiate the detection area and thereby generate a reflected light from the detection area; and the reflected light guiding structure includes a second mirror assembly for altering the direction of the reflected light to provide the output light.

2. The SPR device as disclosed in claim 1, wherein the output light travels substantially in the same direction as the incident light or substantially in the opposite direction from the incident light.

3. The SPR device as disclosed in claim 1, wherein the first mirror assembly includes a plane mirror and a curved mirror disposed obliquely opposite each other, or two plane mirrors disposed obliquely opposite each other.

4. The SPR device as disclosed in claim 1, wherein the second mirror assembly includes a plane mirror and a curved mirror disposed obliquely opposite each other, or two plane mirrors disposed obliquely opposite each other.

5. The SPR device as disclosed in claim 1, wherein the reflective section includes a light-transmissive member and an SPR excitation layer covering the light-transmissive member, the light-transmissive member is a glass layer or a plastic glass layer, and the SPR excitation layer includes a metal film, wherein the metal film includes at least one film selected from a group consisting of a gold film, a silver film, an aluminum film, and a copper film.

6. The SPR device as disclosed in claim 5, wherein an oxidation-resistant film is further formed on a surface of the metal film.

7. The SPR device as disclosed in claim 1, further comprising a processor; when in operation, a microfluidic chip containing a microfluidic sample to be tested is installed in the detection area, and the processor obtains a signal parameter of the reflected light from the optical detector and performs a conversion operation on the signal parameter to determine at least one physical or chemical characteristic value of at least one type of molecules in the microfluidic sample, wherein the conversion operation is a lookup table operation.

8. The SPR device as disclosed in claim 7, wherein the at least one type of molecules is selected from a group consisting of proteins, small molecules, nucleic acids, bioactive molecules, intact cells, viral particles and bacteria.

9. The SPR device as disclosed in claim 7, wherein the at least one physical or chemical characteristic value includes a concentration of one type of the at least one type of molecules and/or a binding rate constant or a dissociation rate constant between two types of the at least one type of molecules.

10. The SPR device as disclosed in claim 7, wherein the signal parameter is selected from a group consisting of a light intensity parameter, a light phase parameter, a resonance angle parameter, a resonance wavelength parameter and an interference pattern parameter.
